(19) United States
(12) Patent Application Publication
(10) Pub. No.: US 2016/0081808 A1
(43) Pub. Date: Mar. 24, 2016

(54) SYSTEMS AND METHODS FOR FACILITATING SURGICAL PROCEDURES INVOLVING CUSTOM MEDICAL IMPLANTS

(71) Applicant: Smith & Nephew, Inc., Memphis, TN (US)

(72) Inventor: Daniel L. McCombs, Paw Paw, MI (US)

(21) Appl. No.: 14/951,602

(22) Filed: Nov. 25, 2015

Related U.S. Application Data
(63) Continuation of application No. 13/617,075, filed on Sep. 14, 2012.
Application No. 13/617,075 is a continuation of application No. 11/593,294, filed on Nov. 6, 2006.
(60) Provisional application No. 60/733,847, filed on Nov. 4, 2005.

(51) Int. Cl. A61F 2/30; A61B 17/16; A61B 19/00

(52) U.S. Cl. CPC A61F 2/30942; A61B 19/50; A61B 17/16; A61B 2019/507; A61B 2019/508

(57) ABSTRACT

Systems and methods for planning surgical procedures involving custom medical implants that can involve the selection, design and/or creation of custom medical implants and/or the selection, modification, and/or design of custom surgical procedures related to those implants. Certain embodiments allow implants to be selected, designed, created, or otherwise customized and then placed in patients using non-standard surgical techniques. The invention provides greater flexibility in implant use by allowing a surgeon to revise, create, or otherwise select surgical techniques for custom implants and, ultimately, provide better treatment in a greater variety of medical circumstances. Certain embodiments of the invention involve computer assisted surgery to provide for implant-related surgery involving non-standard surgical steps and/or implants.

### RELATED APPLICATIONS

This invention claims priority to U.S. provisional patent application No. 60/733,847, entitled “CUSTOM KNEE IMPLANTS ENABLED THROUGH COMPUTER ASSISTED SURGERY (CAS),” filed on Nov. 4, 2005, the entirety of which is hereby incorporated by reference.

### RELATED FIELDS

This invention relates to systems and methods for designing, planning, and otherwise facilitating or enabling surgical procedures involving medical devices, such as biomedical implants, including the design, creation, and surgical implanting of an implant.

### BACKGROUND OF THE INVENTION

Surgeons routinely implant biomedical devices, including but not limited to intramedullary nails and replacement joint parts, into patients. Such biomedical implants are often provided by medical implant manufacturers, who may offer a number of standard, i.e., non-custom, sizes and designs. While such standard implants are designed to be appropriate for a variety of patient conditions, in some cases, standard implants are not appropriate, and a custom implant must be created or an alternative treatment employed. Examples of situations in which custom implant use is appropriate are provided in U.S. patent application Ser. No. 11/506,575 entitled “SYSTEM FOR BIOMEDICAL IMPLANT CREATION AND PROCUREMENT,” the entirety of which is hereby incorporated by reference. Customized implants provide the flexibility of selecting an implant with an appropriate design to treat the patient's condition. However, various problems arise in the context of traditional custom implant development and use. For example, communication between surgeons and medical device companies with respect to the design of the custom implant is often not optimal, collaborative, or otherwise as effective as is possible.

Custom implant design and use is also limited because an implant's design may depend on the associated surgical procedure, and vice versa. For example, in the context of arthroplasty (knee replacement) surgery, typically, standard implant parts are designed to interact with bone resections surgically made in locations and orientations appropriate for the implant parts. More specifically, these surgeries often involve resurfacing the knee joint by replacing a portion of the femur (thigh bone), tibia (shinbone), and/or patella (knee cap) with medical implants, which may be cemented or otherwise attached to the remaining portions of resected bone. Attachment of the implant, and therefore implant design, may in some cases thus depend on the altered anatomic structures created by resections and other surgical techniques.

Differences in both a patient's anatomy and details of an injury make every application of a surgical implant unique to some extent. In some cases, for example, the surgical site's condition (bone condition, surgical access, etc.) is not ideal or even suitable for resections in the standard locations and/or orientations. While various surgical techniques may be used to allow the use of standard implant parts in such non-ideal circumstances, e.g., using bone graft to help correct bone deficiencies, it would often be preferable to perform a “custom” surgery (e.g., cutting in non-standard locations and orientations) using a custom implant (e.g., one capable of attaching to the non-standard bone resection(s)). However, use of custom implants that requires non-standard cuts is hindered by several factors. For example, such efforts may be prevented by a lack of coordination in creating and using a custom implant in a “custom” surgical procedure. Such efforts may depend in part on coordinating information about the patient's physiology, the sensitivity of the surgical procedure to changes in technique and implant attributes, and the sensitivity of the medical device's performance to changes in implant attributes and surgical procedure. In addition, performing a non-standard surgical procedure (e.g., making non-standard cuts to accommodate a custom implant) may require additional knowledge or skill on the part of a performing surgeon or require different, or additional, medical equipment (guides, cutting blocks, etc). There is a need for additional systems and methods that provide for, or otherwise facilitate, designing, planning, and/or otherwise enabling non-standard surgical procedures involving custom medical devices.

### SUMMARY OF THE INVENTION

Embodiments of the invention provide systems and methods for planning surgical procedures involving medical implants. Certain embodiments involve the selection, design and/or creation of custom medical implants and/or the selection, modification, and/or design of custom surgical procedures related to those implants. Certain embodiments allow implants to be selected, designed, created, or otherwise customized and then placed in patients using surgical techniques that are different from the techniques used with standard medical implants or that otherwise alter the structure of the patient's anatomy to have attributes that allow the implant to be attached.

It is accordingly an aspect of some embodiments of the invention to provide systems and methods that provide greater flexibility in implant use by allowing a surgeon and/or others to revise, create, or otherwise select surgical techniques for custom implants and, ultimately, provide better treatment in a greater variety of medical circumstances.

Certain embodiments of the invention involve computer aided surgery (CAS) systems and methods. Certain embodiments provide to a CAS system information regarding a surgery and/or an implant, either or both of which may have custom components. For example, the CAS system may use 3D model geometry to display an image representative of an actual custom implant being used. Alternatively, the preplanning stage of a CAS surgery and the design of a custom implant and associated custom surgical steps may be combined in a combined planning process that allows a surgeon to design and plan the CAS surgery using a single integrated tool. In either case, the CAS system is then used to facilitate the surgical procedure.

It is accordingly an aspect of some embodiments of the invention to provide systems and methods that provide computer-controlled modifications to implant surgical procedure to accommodate non-standard implants and/or non-standard surgical procedures in conjunction with associated CAS systems and processes.

Other aspects, features, and advantages of embodiments of the invention will become apparent with respect to the remainder of this document.

### GENERAL DESCRIPTION OF THE DRAWINGS

FIG. 1 illustrates an exemplary design and CAS system according to certain embodiments of the invention.

FIG. 2 is an exemplary implant illustrating non-standard geometry according to certain embodiments of the invention.

### DETAILED DESCRIPTION OF THE DRAWINGS

While the following exemplary embodiments relate to procedures and implants for knee arthroplasty, the invention is not limited to any particular surgical technique or medical device type. The embodiments illustrate general principles that are applicable for a variety of surgical techniques and medical devices.

Certain exemplary embodiments involve planning and/or executing an arthroplasty surgical procedure. The arthroplasty surgical procedure plan may utilize information received from a surgeon or other person about the surgical site, including, for example, the location and/or orientation of resection cuts. The surgeon or other person may make such selections, for example, based on information about bone quality and the articular geometry of the surgical site. The surgeon and/or other persons may further participate in the selection or design of aspects of custom medical implant designed for use in the planned surgical procedure. So, for example, a surgeon may specify a custom implant that would have “box cuts” positioned differently than otherwise and be able to adjust, in the surgical plan, the target location for the cuts that allow proper placement of the component.

Accordingly, one embodiment involves a method of planning and executing a medical implant surgical procedure that may involve receiving information about a surgical site of a patient. The method may further involve using the information about the surgical site to determine a surgical procedure step for creating an anatomic structure attribute that allows attachment of a medical implant. The method may further involve using information about the anatomic structure attribute to determine a custom attribute of the medical implant for attaching the medical implant to the anatomic structure attribute. The method may further involve creating the medical implant comprising the custom attribute and implanting the medical implant at the surgical site by (i) following the surgical procedure step and (ii) attaching the medical implant custom attribute to the anatomic structure attribute.

The method may involve estimating implant performance after surgeries given potential combinations of potential medical implant attributes and potential anatomic structure attributes and/or determinations of additional medical equipment required in surgeries involving potential combinations of the potential medical implant attributes and potential anatomic structure attributes. The method may involve estimating the difficulty of surgeries involving potential combinations of the potential medical implant attributes and potential anatomic structure attributes and/or estimations of the minimum levels of skill required of a surgeon in surgeries involving potential combinations of the potential medical implant attributes and potential anatomic structure attributes.

Certain embodiments of the invention provide systems for planning a medical implant surgical procedure. Such systems may involve components that allow users (from surgeons to medical device manufacturing specialists to finite element analysis experts and others) to enter information, share information, or otherwise collaborate or contribute to the planning and execution of a medical procedure and provision of an appropriate medical implant. For example, one embodiment involves a computer system comprising a user interface that (1) allows a user, such as a surgeon, to enter information about a surgical site, and (2) a display component that provides an image representative of a custom attribute of a medical implant for allowing attachment at the surgical site and an image representative of an attribute of an altered anatomic structure attribute for attachment of a the custom attribute of the medical implant. The system may allow the user to make adjustments that result in changes to the image representative of the custom attribute of the medical device and the image representative of the attribute of the altered anatomic structure. Changes made using the user interface to one image (i.e. the implant or the anatomy) may change both the image of the implant and the image of the anatomy, i.e., automatically adjusting one based on the change made to the other.

The user interface may further allow medical device manufacturing specialists and others to participate in the planning process and/or contribute by providing information about the sensitivity of the surgical procedure to changes in technique and implant dimensions, the sensitivity of the implant's performance to changes in implant dimensions and surgical procedure, and/or the feasibility of the combination with respect to level of skill of the surgeon and additional required medical equipment, (guides, cutting blocks, etc). Finite element analysis and/or other modeling or simulation techniques may also or alternatively be used to generate one or more proposed device/anatomy combinations.

Certain embodiments involve computer aided surgery. Data, including information regarding the surgery and custom device (e.g., custom cuts and/or custom implants), is provided to a CAS system. In addition, this information could also contain 3D model geometry for use by the CAS system to display an image representative of an actual custom implant being used. During surgery, the CAS system facilitates the placement of the “custom cuts” using standard or alternative instrumentation, typically computer controlled and not encumbered by mechanical alignment means. Thus, one major advantage is that slight computer controlled modifications to cut location and/or orientation may allow arthroplasty for patients with larger deformities that might otherwise be contraindicated. Systems and methods in accordance with these embodiments could thus facilitate a primary knee system.

For example, the method of one embodiment may involve a method of planning and executing a medical implant surgical procedure involving receiving input relating to an attribute of an anatomic structure for attachment of an implant. The method may further involve using the input to determine a custom attribute of the implant for attachment to the anatomic structure attribute. The method may further involve providing to a CAS system (i) information about the custom implant attribute and (ii) information about the anatomic structure attribute. The method may further involve providing during surgery from the CAS system (i) an image representative of the custom implant attribute and (ii) an image representative of the anatomic structure attribute. The images may overlap, be adjacent or otherwise be available for viewing or use by the surgeon. The CAS system may further provide information regarding surgical steps associated with surgically modifying the patient's anatomy to have the designed physical attributes using images or information about the attributes from a pre-surgery plan.

### Exemplary Design Components

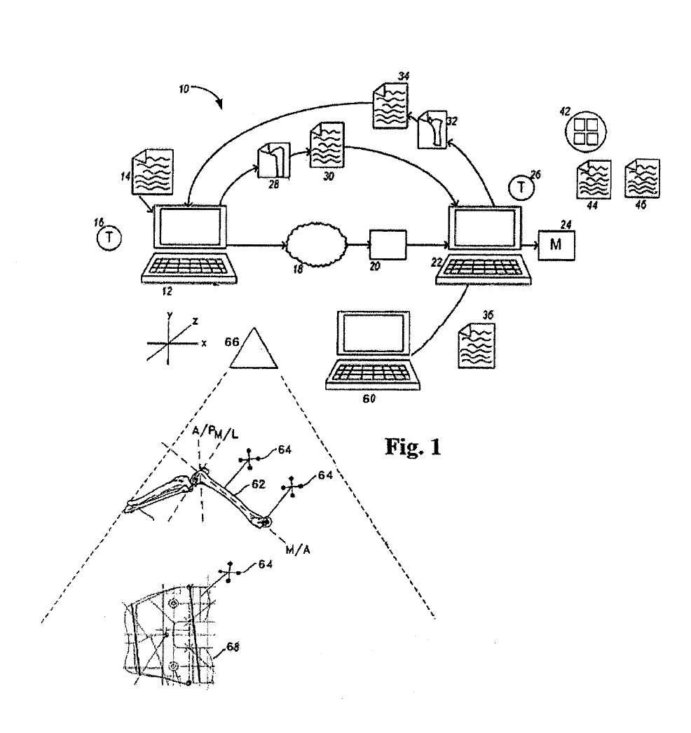

Referring now to FIG. 1, system 10 of certain embodiments can be provided with a surgeon's design device 12. In FIG. 1, the surgeon's design device 12 is embodied as a computer, but other embodiments can include a facsimile machine, a telephone, a handheld computing device, or a pager. The surgeon's design device 12 can access a tool 16 to input information 14. The tool 16 can be provided on the surgeon's design device 12, or can be provided on the automatic platform 22 or the server 20 and can be accessible by the surgeon's design device 12. Information 14 can include identifying information for the surgeon and/or the patient, including names, case numbers, and insurance information, if desired. In certain embodiments, information 14 can include diagnostic data, such as a digital X-ray image, a magnetic resonance image (“MRI”), or a computer tomography (“CT”) image. The surgeon's design device 12 can access other tools 16 that can be provided on the surgeon's design device 12, or can be provided on the automatic platform 22 or the server 20 and can be accessible by the surgeon's design device 12. One tool 16 can enable the surgeon to send a request 28 for a template 32 that represents a biomedical implant, an accessory to a biomedical implant, and/or represents one or more exemplary anatomic structures, features, or attributes. Such a tool 16 may provide the surgeon with various choices, such as a choice of implant type.

Certain embodiments also provide tools 16 for editing at least one design parameter or attribute of a surgical implant template 32. The tools 16 can be provided on the surgeon's design device 12, or can be provided on the automatic platform 22 or the server 20 and can be accessible by the surgeon's design device 12. The tool 16 can further include one or more forms to input one or more design parameters.

Certain embodiments provide tools 16 for editing at least one potential design parameter or attribute of a surgical site altered for attachment of a surgical implant. As with other tools, such a surgical site design tool can be provided on the automatic platform 22 or the server 20 and can be accessible by the surgeon's design device 12 and can include one or more forms to input one or more design parameters. For example, a form embodied as a menu may allow a surgeon to click on the down arrow to obtain a list of design parameters and attributes, and then click to select the desired design parameter of his/her choice. A dimensional reference can serve as a guide while the surgeon is editing a template. Such a reference can help a surgeon to identify design parameters and attributes while he/she is editing a template.

Other embodiments of the surgeon's design device 12 also access tools 16 for creating an implant or altered surgical site design based on diagnostic data 14. As discussed above, diagnostic data 14 can include, among other things, X-rays, MRI images, or CT images. In some embodiments, the tools 16 can enable the surgeon to select certain points or elements associated with the diagnostic data 14. The automated platform 22 can be provided with tools 26 to translate the selected points into a design. In some embodiments, a working model of an implant and/or an altered surgical site can be provided on the surgeon's design device 12 to allow the surgeon to view progress, catch potential mistakes, and edit the design.

Certain embodiments can include a server 20. The server 20 can be embodied as a computer, if desired. The server 20 can be enabled to receive and transmit information between at least the surgeon's design device 12 and the automated platform 22. The server 20 can transmit information over a network 18, which can be embodied as the Internet, or as an Intranet. Other embodiments can include an automated platform 22. The automated platform 22 can be accessed by a biomedical implant manufacturer to practice methods of the invention, In FIG. 1, the automated platform 22 is embodied as a computer, but other embodiments are also within the scope of the invention. The, server 20 and the automated platform 22 can be housed in the same location, or can be housed in different locations, if desired. In the embodiment shown in FIG. 1, the server 20 and the automated platform 22 are two separate elements, but in other embodiments, the server 20 and the automated platform 22 can be members of the same element. In some embodiments, memory 24 can be provided. The memory 24 can be a hard drive of a computer, if desired, but other embodiments are within the scope of the invention. The automated platform 22 can access the memory 24, but the automated platform 22 and the memory 24 need not be members of the same element. The memory 24 can store multiple types of information, including a collection of templates 42, ranges of acceptable design parameters and attributes for implants and associated anatomic structures 44, and/or designs of standard implants and associated anatomic structures for attachment 46.

Embodiments of the automated platform 22 can be provided with tools 26 to automatically generate certain responses to the edits 30 received from the surgeon's design device 12. In some embodiments, the tools 26 can include computer aided design (“CAD”) systems. Common CAD systems known in the art can include SolidWorks®, produced by SolidWorks Corporation, 300 Baker Avenue, Concord, Mass. 01742, or Pro Engineer®, produced by Parametric Technology Corporation, 140 Kendrick Street, Needham, Mass. 02494. The CAD systems can be enabled to translate edits 30 of the template 32 received from the surgeon's design device 12 into a design for an implant 34 or altered surgical site.

In some embodiments, the automated platform 22 can have tools 26 to ensure that the design is acceptable for treatment of a patient's condition. Not every type of biomedical implant is appropriate for insertion into the human body, and sometimes the surgeon might not be aware of whether a particular design is acceptable. For example, a regulatory agency, such as the Food and Drug Administration, might have determined that a hip stem cannot exceed a certain weight. The surgeon might not be aware of the ranges of acceptable weights, and might have designed an implant with an unacceptable weight. Similarly, certain injuries or anatomical conditions may make the use of certain implants unacceptable. Accordingly, tools 26 can be provided to ensure that the design of an implant and/or associated altered anatomic attributes are acceptable for treatment of a patient's condition.

In some embodiments there can be ranges of acceptable design parameters 44 to identify whether an edit 30 is acceptable. This informs the surgeon that the edit 30 is not acceptable, and can provide the surgeon with an opportunity to enter another edit. In some embodiments, the automated platform 22 is further enabled to repeat the process as needed by comparing any successive edits to the ranges of acceptable design parameters 44. If the edit 30 is acceptable, then in some embodiments the automated platform 22 can create a final design for the biomedical implant, or can provide the surgeon with an opportunity to enter another edit. Accordingly, in some embodiments, the automated platform 22 can compare an edit 30 to ranges of acceptable design parameters 44.

Alternatively, in some embodiments, the automated platform 22 can have tools 26 to compare a surgeon's design to the standard designs 46. Biomedical implant manufacturers have available a wide variety of standard biomedical implants that attach to a variety of anatomic structures. The surgeon might not be aware that a standard implant is suitable for his/her application. The surgeon might find it desirable to obtain a standard implant because a standard implant is less expensive and more readily available. Accordingly, tools 26 can be provided to practice a method to notify the surgeon that a standard implant might be acceptable for the application. In certain embodiments, the automated platform 22 is further enabled to generate manufacturing instructions (e.g., CAM instructions) that may be automatically sent to a manufacturing facility.

### Exemplary Computer Assisted Surgery Components

FIG. 1 further illustrates computer assisted surgery components 60, 62, 64, 66, 68 that can receive surgical implant and surgical procedure information 36 from the other system components, e.g., from the automated platform 22, for use during a surgical procedure. This information 36 can include information about attributes of a proposed implant 68 as well as information about attributes of the proposed altered anatomy 62 to which the implant 68 is intended to be attached. In certain embodiments, the computer assisted surgery system 60 allows preoperative planning that utilizes this information 36 in which the surgeon selects reference points and determines implant position. The preoperative CAS planning may occur in conjunction with the design of the implant 68 and/or the design of the altered anatomic structures 62 for attachment of the implant 68.

Certain embodiments involve computer assisted surgery for tracking anatomy 62, implants 68, instrumentation (not shown), virtual constructs (not shown), rendering images shown, for example, on a monitor of system 60, and data (not shown) related to them in connection with the surgical operation in which anatomic structures 62 are altered (e.g., by resection) and implants 68 are attached to the altered anatomic structures 62. Anatomical structures 62 and various items (e.g., implants 68) may be attached to or otherwise associated with fiducial functionality 64, and constructs (not shown) may be registered in position using fiducial functionality 64 whose position and orientation can be sensed and tracked. Such structures 62, items 68 and constructs can be rendered onscreen properly positioned and oriented relative to each other using associated image files, data files, image input, other sensory input, and/or based on the tracking: This allows surgeons to navigate and perform surgeries using images that reveal interior portions of the body combined with computer generated or transmitted images that show surgical implants 68 and/or other devices located, oriented, and/or attached properly to the body parts 62.

The use of a CAS system may allow more accurate and effective resection of bone, placement and assessment of implants and joint performance, and placement and assessment of performance of actual implants and joint performance. Various alignment modules and other structures and processes may allow for coarse and fine alignment of instrumentation and other devices relative to bone for use in connection with the tracking systems, as explained in U.S. Patent Publication 2003/0069591 entitled “COMPUTER ASSISTED KNEE ARTHROPLASTY INSTRUMENTATION, SYSTEMS, AND PROCESSES,” the entirety of which is hereby incorporated by reference.

Intraoperatively, the CAS system 60 may make use of CT scans, MRI data, digitized points on the anatomy, and other images and information and may calibrate patient position to the preoperative plan, such as using a “point cloud” technique, and can use a robot to make bone preparations. Position and/or orientation tracking sensors, such as infrared sensors, acting stereoscopically or otherwise, may be used to track positions of body parts 62, surgery-related items 68, and virtual constructs or references such as rotational axes which have been calculated and stored based on designation of bone landmarks. Processing capability, such as any desired form of computer functionality, whether standalone, networked, or otherwise, may take into account the position and orientation information as to various items in the position sensing field (which may correspond generally or specifically to all or portions or more than all of the surgical field) based on sensed position and orientation of their associated fiducials 64 or based on stored position and/or orientation information. The processing functionality correlates this position and orientation information for each object with stored information regarding the items, such as a computerized fluoroscopic imaged file of a femur or tibia, a wire frame data file for rendering a representation of an instrumentation component, trial prosthesis or actual prosthesis, or a computer generated file relating to a rotational axis or other virtual construct or reference. The processing functionality then displays position and orientation of these objects on a screen or monitor, or otherwise. Thus, it can display and otherwise output useful data relating to predicted or actual position, orientation, and altered structural attributes of body parts 62, implants 68, and other items and virtual constructs for use in navigation, assessment, and otherwise performing surgery or other operations.

As one example, images such as fluoroscopy images showing internal aspects of the femur and tibia can be displayed on a monitor in combination with actual or predicted shape, position and orientation of surgical implants and altered anatomic structure in order to allow the surgeon to properly position and assess performance of various aspects of the joint being repaired, reconstructed or replaced. The surgeon may use this preoperatively to design an appropriate surgical implant 68 and corresponding altered anatomic structure attributes. The surgeon may then interoperatively use navigation tools, instrumentation, trial prostheses, actual prostheses and other items relative to bones and other body parts in order to perform surgeries more accurately, efficiently, and with better alignment and stability. This system may also generate data based on position tracking and, if desired, other information to provide cues on screen, aurally or as otherwise desired to assist in the surgery such as suggesting certain surgical steps in accordance with a predefined surgical plan, e.g., bone modification steps. Moreover, interoperatively such bone modification steps may be modified based on actual surgical conditions, e.g., automatically requiring or suggesting that a surgeon release certain ligaments or portions of them based on the actual performance of components as sensed by the instrumentation, systems, and processes of the CAS system.

### Exemplary Design of Surgical Device for Custom Surgery

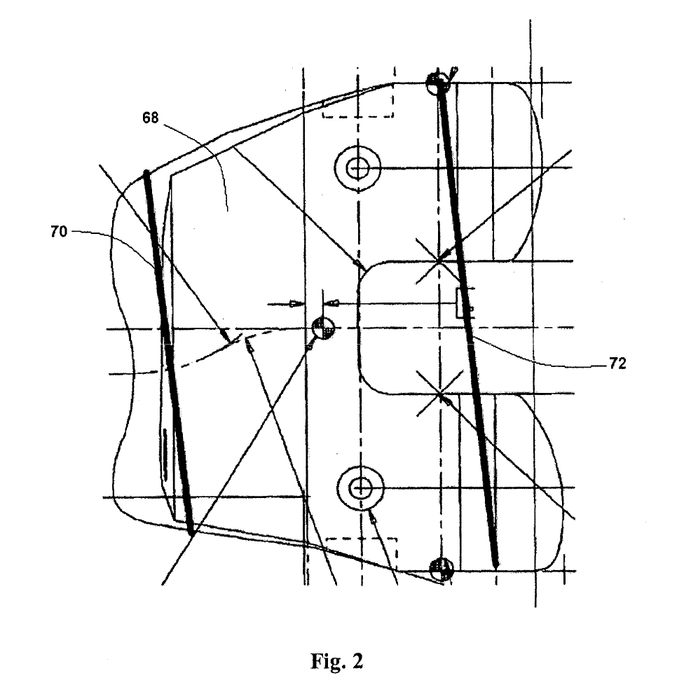

Referring now to FIG. 2 of an exemplary implant illustrating non-standard geometry according to certain embodiments of the invention. Specifically, a femoral knee implant 68 is illustrated having attributes 70, 72 presenting non-standard geometries for attachment to a femur bone with appropriate resections. This custom implant 68 is therefore capable of attaching to bones that cannot or should not be resected in standard resection locations, expanding the medical circumstances in which a surgical implant may be used.

The foregoing is provided for purposes of illustration and disclosure of a preferred embodiment of the invention. Changes, deletions, additions, and modifications may be made to the structures disclosed above without departing from the scope or spirit of the present invention.

1. A method of planning and executing a medical implant surgical procedure comprising:
a) receiving information about a surgical site of a patient;
b) using the information about the surgical site to determine a surgical procedure step for creating an anatomic structure attribute that allows attachment of a medical implant;
c) using information about the anatomic structure attribute to determine a custom attribute of the medical implant for attaching the medical implant to the anatomic structure attribute;
d) creating the medical implant comprising the custom attribute; and
e) implanting the medical implant at the surgical site by (i) following the surgical procedure step and (ii) attaching the medical implant custom attribute to the anatomic structure attribute.

2. The method of claim 1 wherein using information about the anatomic structure attribute to determine a custom attribute of the medical implant further comprises estimations of implant performance after surgeries.

3. The method of claim 1 wherein using the information about the surgical site to determine the surgical procedure step further comprises estimations of the difficulty of the surgical procedure step.

4. The method of claim 1 wherein using the information about the surgical site to determine the surgical procedure step further comprises estimations of a minimum level of skill required to perform the surgical procedure step.

5. A system for planning a medical implant surgical procedure comprising:
a) a user interface that allows a user to enter information about a surgical site; and
b) a display component that provides (i) an image representative of a custom attribute of a medical implant for allowing attachment at the surgical site and (ii) an image representative of an altered anatomic structure attribute for attachment of the custom attribute of the medical implant;
wherein the system allows the user to make adjustments that result in changes to the image representative of the custom attribute of the medical device and the image representative of the attribute of the altered anatomic structure.

6. The system of claim 5 wherein the image representative of the custom attribute of the medical device and the image representative of the attribute of the altered anatomic structure are displayed overlapping one another.

7. The system of claim 5 wherein the image representative of the custom attribute of the medical device and the image representative of the attribute of the altered anatomic structure are displayed adjacent to one another.

8. The system of claim 5 wherein the system allows the user to make adjustment through the user interface, wherein the user interface allows the user to graphically make adjustments.

9. The system of claim 5 wherein user adjustments are constrained by estimations of implant performance.

10. A method of planning and executing a medical implant surgical procedure comprising:
a) receiving input relating to an attribute of an anatomic structure for attachment of an implant;
b) using the input to determine a custom attribute of the implant for attachment to the anatomic structure attribute;
c) providing to a CAS system (i) information about the custom implant attribute and (ii) information about the anatomic structure attribute; and
d) providing during surgery from the CAS system (i) an image representative of the custom implant attribute and (ii) an image representative of the anatomic structure attribute.

11. The method of claim 10 wherein the image representative of the custom implant attribute overlaps the image representative of the anatomic structure attribute.

12. The method of claim 10 wherein the image representative of the custom implant attribute is adjacent to the image representative of the anatomic structure attribute.

13. The method of claim 10 wherein the CAS system further provides information regarding surgical steps associated with creating a surgical site anatomy according to the image representative of the anatomic structure attribute.
